(19) United States
(12) Patent Application Publication
(10) Pub. No.: US 2005/0181353 A1
(43) Pub. Date: Aug. 18, 2005

(54) Stabilization of cells and biological specimens for analysis

(76) Inventors: Galla Chandra Rao, Princeton Junction, NJ (US); Melissa Herman, Hatboro, PA (US); Herman Rutner, Hatboro, PA (US); Leon W.M.M. Terstappen, Huntingdon Valley, PA (US)

Correspondence Address: IMMUNICON CORPORATION, 3401 MASONS MILL ROAD, SUITE 100, HUNTINGDON VALLEY, PA, 19006, US

(21) Appl. No.: 10/780,349

(22) Filed: Feb. 17, 2004

(51) Int. Cl. A01N 1/02; A61K 31/727; A61K 31/198

(52) U.S. Cl. 435/1.1; 435/2; 514/56; 514/566

(57) ABSTRACT

Compositions and methods for stabilizing rare cells in blood specimens, preserving the quality of blood specimens, and also serving as cell fixatives are disclosed which minimize losses of target cells (for example, circulating tumor cells) and formation of debris and aggregates from target cells, non-target cells and plasma components, thereby allowing more accurate analysis and classification of circulating tumor cells (CTC) and, ultimately, of tumor burdens in cancer patients. Stabilization of specimens is particularly desirable in protocols requiring rare cell enrichment from blood specimens drawn from cancer patients. Exposure of such specimens to potentially stressful conditions encountered, for example, in normal processing, mixing, shaking, delays due to transporting the blood, has been observed to not only diminish the number of CTC but also to generate debris and aggregates in the blood specimens that were found to interfere with accurate enumeration of target cells, if present. Stabilizers are necessary to discriminate between in vivo CTC disintegration and in vitro sample degredation.

### PRIORITY INFORMATION

This application claims priority under 35 USC §119(e) to U.S. Provisional Applications No. 60/314,151 filed 23 Aug. 2001, and No. 60/369,628 filed 3 Apr. 2002. Both of these applications are incorporated by reference herein.

### FIELD OF THE INVENTION

This invention relates generally to the field of cell and blood stabilization, more particularly to the stabilization of rare cells in blood specimens and most particularly to stabilization of circulating tumor cells (CTC) in whole blood for subsequent enrichment and analysis.

### BACKGROUND OF THE INVENTION

Tumor cells were detected in blood as early as 1869. There is evidence that primary cancers begin shedding neoplastic cells into the circulation at an early disease stage prior to the appearance of clinical manifestations. Upon vascularization of a tumor, tumor cells shed into the circulation may attach and colonize at distant sites to form metastases. These circulating tumor cells are not normally found in healthy individuals and thus can form the basis for diagnosis and treatment of specific carcinomas. Neo-vascularization takes place when the tumor grows to a diameter of 1-2 mm, a size too small to be detected by conventional methods such as mammography, which requires a tumor size of approximately 5 mm for detection. A test method that has the sensitivity and specificity to detect small numbers of CTC at an earlier stage than the current gold standard, mammography, could dramatically improve early-stage cancer diagnosis and disease management. Such a test is taught in U.S. Pat. No. 6,365,362 by Terstappen et al., and is incorporated by reference herein.

Whole blood is a complex body fluid containing diverse populations of cellular and soluble components capable of undergoing numerous biochemical and enzymatic reactions that may occur particularly on prolonged storage for more than 6 hours in vivo, herein defined as occurring in the patient's body, and in vitro, herein defined as occurring after blood draw. Some of these reactions are directed to destruction of circulating tumor cells as foreign species. The patient's immune response further weakens or destroys tumor cells by the normal defense mechanisms including phagocytosis and neutrophil activation. Chemotherapy similarly is intended to reduce both cell function and proliferation by inducing cell death by necrosis.

Besides these external destructive factors, tumor cells damaged in a hostile environment may undergo programmed death or apoptosis. Cells undergoing apoptosis or necrosis have altered membrane permeabilities, thereby allowing escape of DNA, RNA, and other cellular components leading to formation of cellular debris and eventual complete disintegration of CTC. Such tumor cell debris may still bear epitopes that are characteristic of intact cells, and can lead to spurious increases in circulating cancer cells. Even whole blood specimens from healthy individuals undergo substantial changes in the cellular composition, broadly categorized and herein defined as decreased blood quality, which may occur with prolonged storage for periods of greater than 24 hours. Erythrocytes may rupture and release hemoglobin and produce cell ghosts. Leukocytes, particularly granulocytes, are known to be labile and diminish on storage. Such changes increase the amount of cellular debris, derived from normal blood cells or proteins were found to interfere with the isolation and detection of rare target cells such as CTC. The combined effects of these destructive processes show a substantial increase in cellular debris that is readily detectable, for instance, with flow cytometric and microscopic analyses. Methods for such analysis are described in a commonly owned, co-pending application entitled “Analysis of circulating tumor cells, fragments, and debris,” which is incorporated by reference herein.

Detection of circulating tumor cells by microscopic imaging is similarly adversely affected by spurious decreases in classifiable tumor cells and a corresponding increase in interfering stainable debris. Hence, maintaining the integrity or the quality of the blood specimen is of utmost importance, since there may be a delay of as much as 24 hours between blood draw and specimen processing.

Such delays are quite common, since the techniques and equipment used in processing blood for this assay may not be readily available in every laboratory. The time necessary for a sample to arrive at a laboratory for sample processing may vary considerably. It is therefore important to establish the time window within which a sample can be processed. In routine hematology analyses, blood samples can be analyzed within 24 hours. However, as the analysis of rare blood cells is more critical, the time window in which a blood sample can be analyzed shortens. An example is immunophenotyping of blood cells, which, in general, has to take place within 24 hours. In a cancer blood assay, larger volumes of blood have to be processed, and degradation of the blood sample can become more problematic as materials released by disintegrating cells can increase the background and, therefore, decrease the ability to detect tumor cells.

There is a large body of published or patented art regarding the stability and stabilization of normal blood cells over time and several proprietary commercial stabilizers are available for preserving white blood cells, e.g. Cyto-Chex™ from Streck Laboratories, Omaha, Nebr., StabilCyte™ from BioErgonomics, St. Paul, Minn., and TRANSfix™ from UK NEQAS, Sheffield, UK.

In WO 97/45729, TRANSfix™ stabilizer is claimed to be suitable for analysis of pathological specimens, specifically for HIV and leukemia blood specimens. However, no data are shown for these applications or for the use of TRANSfix™ stabilizer for stabilizing or protecting CTC during storage for prolonged periods. TRANSfix™ stabilizer is claimed to contain the crosslinking fixative, paraformaldehyde and heavy metal ions. It was shown to preserve the integrity of leukocytes including granulocytes for at least 5 days as measured by flow cytometry. No data are shown for CTC or other pathogens.

Despite the shortcomings of paraformaldehyde or reagents containing paraformaldehyde, formaldehyde, glutaraldehyde and glyoxal, such reagents are frequently used for fixing and stabilizing tumor cells in blood or histology specimens (see, for example, D. B. Tse et al., U.S. Pat. No. 6,004,762). Effective fixing is particularly important after CTC have been permeabilized with pore-forming reagents, such as saponin or surfactants, which further weaken the membrane structure and integrity of the fragile CTC. Permeabilization is required to permit staining or immunostaining of intracellular elements, for example, with the nuclear stain DAPI (4,6-diamidino-2-phenylindole) and with labeled antibodies, such as for cytokeratins, which are used for characterizing CTC and differentiating them from normal blood cells.

Alternative fixatives to bifunctional or crosslinking aldehydes have been used. Some of the older fixatives are based on heavy metals, e.g. chromium or manganese, similar to the mode of action in tanning of leather hides, but their lack of specificity and toxicity limits applications. Another approach to fixation utilizes monofunctional derivatives of formaldehyde, or methylol derivatives of heterocyclic amines or amides, e.g. diazolinidinyl urea and imidazolidinyl urea that are widely used in cosmetics as preservatives. Also, polyethylene glycol (of about 20,000MW) is described as being an effective stabilizing agent for leukocytes. Such compounds are disclosed in several US patents issued to Streck Laboratories, Omaha, Nebr., (U.S. Pat. Nos. 5,459,073; 5,849,517; 5,981,282; 6,017,764; 6,051,433; 6,124,089; 6,159,682; 6,200,500) which are primarily intended for stabilizing specific blood cell populations for use as hematology controls.

The mode of action of methylol derivatives is unknown but is speculated to involve weak, reversible bonds with amino groups of cellular proteins, which may dissociate upon removal of the excess fixative. Methylol or hydroxymethyl derivatives are chemically labile and may release small amounts of formaldehyde that could form short-range or single carbon crosslinks with proteins. Formaldehyde released from these so-called formaldehyde donors has been reported to react with nucleic acid bases, particularly adenine, to reversibly form hydroxylmethylol derivatives and methylene bridges thereby irreversibly crosslinking nucleic acids, which may be the biocidal mode of action in cosmetics. However, free formaldehyde is claimed not to be the active ingredient in the Cyto-Chex™ stabilizers in the foregoing patents. These patents do not disclose utility for stabilizing or fixing CTC in blood or other biological specimens. One cannot presume that tumor cells circulating in blood will be stabilized similar to normal blood cells due to the known fragility of CTC, and that any stabilization of CTC, if it does occur, would persist throughout the processing steps. There is therefore a clear need for identifying effective reagents for stabilizing CTC in vitro during storage and processing and for preserving the quality of blood, which we herein have shown to be critical in enrichment and detection procedures requiring accurate classification and enumeration of CTC, if present.

### SUMMARY OF THE INVENTION

Stabilizing agents are necessary to discriminate between in vivo tumor cell disintegration and disintegration due to in vitro sample degradation. In accordance with the present invention, several compositions serving as stabilizers, fixatives, and preservatives for maintaining the quality of biological specimens have been discovered. Also, methods and apparatus for stabilizing biological specimens have been discovered. These improvements or discoveries have enabled the invention described herein to be greatly improved over systems and methods in the art, and to have applications for enrichment and enumeration of CTC in whole blood.

A number of compositions have been discovered to preserve biological specimens. These compositions are a combination of anti-coagulants and stabilizing agents. Further, the invention teaches a composition of a stabilized sample, an anti-coagulant, and a stabilizing agent or agents. Further, the invention teaches various methods for contacting biological specimens with these compositions to enhance stability. Further, the invention teaches various apparatus for contacting biological specimens with these compositions to enhance stability. Generally, these can be used for preserving biological specimens, and specifically for preserving CTC in blood samples.

Accordingly, an improved protocol is provided, which comprises addition of stabilizers to the blood collection tube prior to blood draw. Also provided by the invention are methods for adding the stabilizers to the blood tube immediately after the blood draw, including protocols for compensating for the varying volume of the specimen in the blood tube.

Further provided by the invention are methods for adding the stabilizers to one or more buffers used in processing of the biological specimens thus serving both as stabilizer and/or as a fixative, as required. Accordingly, it is a primary object of the present invention to provide stabilizers for biological specimens prior to analysis. Specific uses of this invention are directed toward stabilizing CTC in blood samples. It is another objective of the invention to provide stabilizers and preservatives for maintaining the quality of whole blood specimens for at least 24 hours, but up to 72 hours, when exposed to mechanical stress, such as may occur during inadvertent mixing or shaking during transport, or during mechanical re-mixing of the specimen prior to analysis.

It is to be understood and appreciated that these discoveries in accordance with the invention are only those that are illustrative of the many additional potential applications of the compositions and methods that may be envisioned by one of ordinary skill in the art, and thus are not in any way intended to be limiting of the scope of the invention. Accordingly, other objects and advantages of the invention will be apparent to those skilled in the art from the following detailed description, together with the appended claims.

### DESCRIPTION OF THE FIGURES

FIG. 1 depicts the decline of detectable CTC in whole blood specimens from 9 cancer patients after storage for 24 hours at room temperature.

FIG. 2 depicts the detectable CTC in whole blood specimens from 3 cancer patients at 0, 6, 18, and 24 hours.

FIG. 3 depicts the effect of Cyto-Chex™ stabilizer on specimen quality after shipping two specimen tubes from one cancer patient, #28162 in Table II: a.) specimen tube without stabilizer, b.) specimen tube with 30% Cyto-Chex™ stabilizer. The images show the staining of the DNA of cells in the enriched sample after adding the nucleic acid dye DAPI. The majority of the round objects represent nuclei inside nucleated cells including CTC, while irregular agglomerates may include DNA released from damaged cells.

FIG. 4 depicts the effect of Cyto-Chex™ stabilizer on specimen quality after standing for 24 hours or mixing on a nutator: a) without mixing and without stabilizer, b) with mixing and without stabilizer, c) with mixing in the presence of 30% Cyto-Chex™ stabilizer. Image analysis after DAPI staining is discussed in FIG. 3b)

FIG. 5 depicts 3 types of CTC degradation: Intact CTC (FIG. 5a), Suspect CTC (FIG. 5b), and “Not Assigned Events” (FIGS. 5c and 5d).

FIG. 6 compares CTC from stabilized blood of 27 subjects at 24 hours and 72 hours showing significantly similar count, as well as the statistical significance for the comparison.

### DETAILED DESCRIPTION OF THE INVENTION

Herein, various terms that are well understood by those of ordinary skill in the art are used. The intended meaning of these terms does not depart from the accepted meaning.

Specimen, sample, or blood quality is herein defined and experimentally determined by the following parameters:
1. relative number of leukocytes that are non-specifically selected as background compared to CTC or spiked cultured tumor cells;
2. cell debris including DNA or DNA fragments as measured by particulate material and larger aggregates that are detectably stained with the nuclear stain DAPI; and
3. cellular cytoplasmic debris or aggregates that are either non-specifically or specifically stained with phycoerythrin (PE) labeled anti-cytokeratin antibodies or with allophycocyanin (APC)-labeled antibody for CD-45 on leukocytes.

The terms “biological specimen” or “biological sample” may be used interchangeably, and refer to a small portion of fluid or tissue taken from a human subject that is suspected to contain cells of interest, and is to be analyzed. A biological specimen may refer to the fluidic or the cellular portions, or to the portion containing soluble material. Biological specimens or biological samples include, without limit bodily fluids, such as peripheral blood, tissue homogenates, nipple aspirates, colonic lavage, sputum, bronchial lavage, and any other source of cells that is obtainable from a human subject. An exemplary tissue homogenate may be obtained from the sentinel node in a breast cancer patient.

The term “rare cells” is defined herein as cells that are not normally present in biological specimens, but may be present as an indicator of an abnormal condition, such as infectious disease, chronic disease, injury, or pregnancy. Rare cells also refer to cells that may be normally present in biological specimens, but are present with a frequency several orders of magnitude less than cells typically present in a normal biological specimen.

The terms “anti-coagulant” or “anti-coagulating agent” may be used interchangeably, and refer to compositions that are added to biological specimens for the purpose of inhibiting any undesired natural or artificial coagulation, agglutination, or aggregation, further collectively defined as “clumping” or “clump formation”. However, such clumps must be differentiated from “clusters” or aggregates of CTC that are counted individually as Intact CTC if they meet the classification criteria for Intact CTC. Clusters of CTC, by virtue of their adhesiveness and propensity to establish secondary metastatic tumor sites, are believed to have greater proliferative potential than single CTC and their presence is thus diagnostically highly significant. An example of coagulation is blood clotting and common anti-coagulants are chelating agents, exemplified by ethylenediamine tetraacetic acid (EDTA), diethylenetriamine pentaacetic acid (DTPA), 1,2-diaminocyclohexane tetraacetic acid (DCTA), ethylenebis(oxyethylenenitrilo) tetraacetic acid (EGTA), or by complexing agents, such as heparin, and heparin species, such as heparin sulfate and low-molecular weight heparins.

The ideal “stabilizer” or “preservative” (herein used interchangeably) is defined as a composition capable of preserving target cells of interest present in a biological specimen, while minimizing the formation of interfering aggregates and/or cellular debris in the biological specimen, which in any way can impede the isolation, detection, and enumeration of targets cells, and their differentiation from non-target cells. In other words, when combined with an anti-coagulating agent, a stabilizing agent should not counteract the anti-coagulating agent's performance. Conversely, the anti-coagulating agent should not interfere with the performance of the stabilizing agent. Additionally, the disclosed stabilizers also serve a third function of fixing, and thereby stabilizing, permeabilized cells, wherein the expressions “permeabilized” or “permeabilization” and “fixing”, “fixed” or “fixation” are used as conventionally defined in cell biology. The description of stabilizing agents herein implies using these agents at appropriate concentrations or amounts, which would be readily apparent to one skilled in cell biology, where the concentration or amount is effective to stabilize the target cells without causing damage. One using the compositions, methods, and apparatus of this invention for the purpose of preserving rare cells would obviously not use them in ways to damage or destroy these same rare cells, and would therefore inherently select appropriate concentrations or amounts. For example, the formaldehyde donor imidazolidinyl urea has been found to be effective at a preferred concentration of 0.1-10%, more preferably at 0.5-5% and most preferably at about 1-3% of the volume of said specimen. An additional agent, such as polyethylene glycol has also been found to be effective, when added at a preferred concentration of about 0.1%-5%, more preferably about 0.1%-1%, and most preferably about 0.1%-0.5% of the specimen volume.

A stabilizing agent must be capable of preserving a sample for at least a few hours. However, in the Examples presented herein, it is shown that samples can be stabilized for at least up to 72 hours. Such long-term stability is important in cases where the sample is obtained in a location that is distant to the location where processing and analysis will occur. Furthermore, the sample must be stabilized against mechanical damage during transport.

The term “circulating tumor cells” or CTC refers to cells known to be shed from tumors, often in large numbers. A steady-state level is maintained when destruction of CTC equals the shedding rate, which in turn depends on the size of the tumor burden (see JG Moreno et al. “Changes in Circulating Carcinoma Cells in Patients with Metastatic Prostate Cancer Correlates with Disease State.” Urology 58. 2001). Generally, the more resistant and proliferative cells survive to establish secondary or metastatic sites. Circulating tumor cells are the preferred target rare cells to be stabilized or fixed by this invention.

The term “cell composition” refers to a plurality of cells, most likely of different types derived from a biological specimen. This cell composition will include both target and non-target cells. For example, in a fraction of blood taken from a subject that is suspected to have cancer, a cell composition would be red blood cells, white blood cells, and circulating tumor cells if present. However, the skilled biologist will recognize that other biological specimens, as described herein, would contain different cell types, depending on the source of the biological specimen.

During methods of enrichment, such as those taught by Terstappen et al. in U.S. Pat No. 6,365,362 (incorporated by reference herein), the enriched cell fraction contains mainly leukocytes and a much smaller number of CTC, if present. During processing, cells are permeabilized with a mild surfactant, Immuniperm™ (PBS containing 0.05% Saponin—Immunicon Corp, Huntingdon Valley, Pa.) to form small pores of about 8 nm diameter to permit entry of the immunostaining antibody, anti-cytokeratin-PE, and the nuclear stain, DAPI. DAPI is a fluorescent intercalating dye that binds selectively to adenine-thymine in cellular and extra-cellular DNA. These pores further weaken the structural integrity of the cell membrane and require a cell fixative or stabilizer after staining to “plug” the holes. As previously mentioned, the conventional aldehyde and heavy metal based fixatives in the art, which are intended to strengthen the permeabilized membrane, were found to be incompatible with the processes of magnetic collection and with the staining protocols used in this invention. In contrast, the Cyto-Chex™ stabilizers used in this invention were also unexpectedly found to be highly effective as fixatives in the post-staining steps and are thus included in the proposed reagent formulation herein. Hence, the stabilizers described and used in this invention concurrently or sequentially were discovered to perform three critical functions:
1. stabilization of CTC and non-target cells in blood specimens for transport, storage, sample preparation, and subsequent analysis,
2. preserving the quality of the blood specimen by inhibiting debris and/or aggregate formation, and
3. serving as a fixative after permeabilization.
   One or more of these functions were found to be essential for accurate analysis of CTC or rare cells in the enrichment assays utilized in this invention.

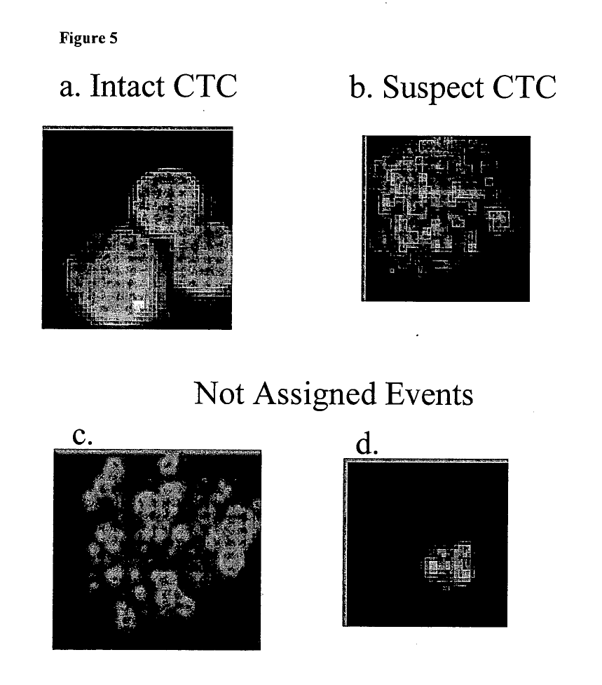

The presence of a stainable nucleus (e.g. stainable by a nuclear dye such as DAPI) is an essential morphological characteristic of a nucleated cell. Positive DAPI staining is a critical element in defining and differentiating cytokeratin-positive cells as “Intact CTC” (FIG. 5a), which are distinct from “Suspect CTC” characterized by DAPI-weak or DAPI-negative and/or irregular or speckled cytoplasmic staining of the cytoskeletal proteins recognized by the anti-cytokeratin antibodies. Both Intact and Suspect CTC can be distinguished from leukocytes that are DAPI-positive, but express the leukocyte-specific marker, CD45. “Suspect CTC” still have some morphological features that can be associated with Intact CTC and may be necrotic, apoptotic cells or “apoptotic bodies” of epithelial cell origin that originated from “Intact CTC” or were shed from the tumor site into the circulation (FIG. 5b).

A third category of particles or detectable events, labeled “Not Assigned Events” in Table II and FIGS. 5c and 5d, can have staining characteristics similar to “Suspect CTC”, but lack structural morphological features of cells, i.e. they are largely irregular aggregates or clumps that were found to be statistically more prevalent in unstabilized compared to stabilized blood specimens (p=0.0327). The presence of a substantial number of “Suspect CTC” relative to “Intact CTC” in a stabilized blood specimen can provide important diagnostic information to the clinician regarding the growth/tumor burden, immune status and/or the therapeutic response of a patient. Table I shows the stabilizing effects of different stabilizers on the relative number of Intact CTC, Suspect CTC, and “Not Assigned Events” in eight cancer patients. Table II shows a significant decrease (p=0.0327) in the number of “Not Assigned Events” with Cyto-Chex™ stabilizer as compared to no stabilizer and the significant stabilizing effect of Cyto-Chex™ stabilizer on CTC as compared to no stabilizer (p=0.0004) in thirty-one patients with diverse carcinomas. The number of Suspect CTC was not statistically different between the stabilized and the not stabilized blood (p=0.1548). The likely cause for the increase in background in the unstabilized blood is the artifacts that cannot be discriminated from “true” Suspect CTC. Discrimination between artifacts and Suspect CTC or tumor debris formed in vivo can have clinical significance and emphasizes the need for a blood/CTC stabilizer that permits the assessment of the CTC status in a patient's blood without contributions from artifacts caused by sample degradation between the blood draw and the CTC analysis (i.e. in vitro).

In the enrichment processes used in the following Examples, CTC (herein generically consisting of both Intact and Suspect CTC, unless differentiated), cell fragments, and cellular debris are captured with magnetically labeled antibodies that recognize specific surface markers on the CTC (for example, with anti-EpCAM antibody attached to magnetic particles of about 0.2 μm (200 nm) diameter, as described in U.S. Pat. No. 6,120,856 which is incorporated by reference herein). The presence of numerous small but dense magnetic particles on the surface of CTC can further stress the weakened non-stabilized cell membrane during the rapid migration of the magnetically labeled cells in the strong magnetic field generated inside a high-gradient magnetic separator (e.g. Immunicon quadrupole QMS17; patented under U.S. Pat. Nos. 5,186,827 and 5,466,574) both during magnetic (in-field) incubation and during magnetic collection.

The stabilizers of this invention were found to inhibit damage to magnetically labeled cells that may occur both from magnetic and non-magnetic stresses even during normal specimen processing, such as in centrifugation, vortexing, and pipetting. For example, substantially greater damage to rare cells and CTC has been observed when using immunomagnetic labeling with the larger magnetic particles or beads of 2.8 μm and 4.5 μm diameters as sold by Dynal Inc., Lake Success, N.Y.

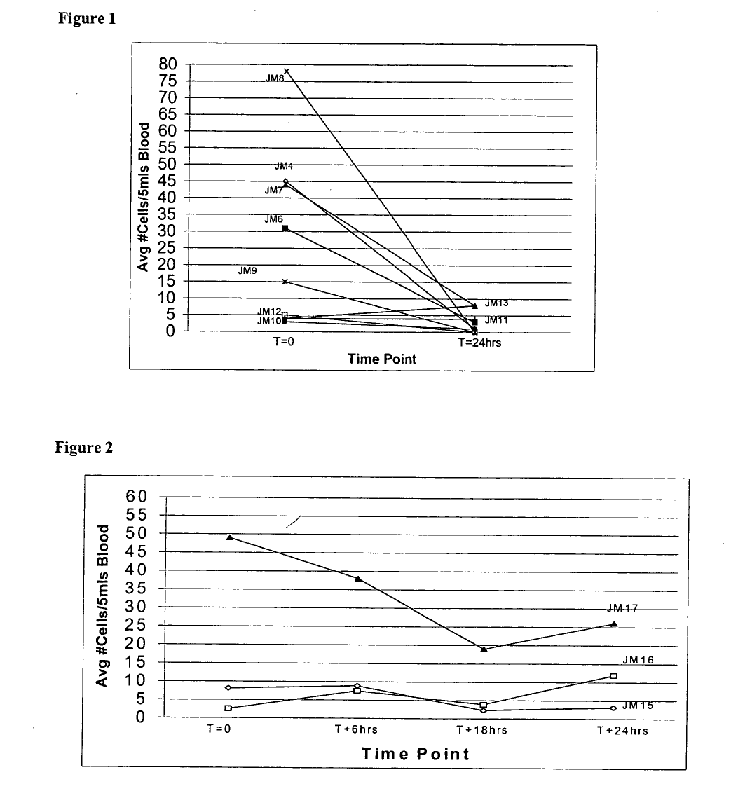

During blood draw and subsequent specimen processing, the surviving battered tumor cells present in the peripheral circulation may be further stressed and damaged by turbulence during blood draw into an evacuated tube and by specimen processing, e.g. transport of the blood tube and mixing prior to analysis. Such mechanical damage is additional to on-going immunological, apoptotic, and necrotic in processes leading to destruction of CTC that occur in vitro in a time dependent manner. We have found that the longer the specimen is stored, the greater the loss of CTC, and the larger the amounts of interfering debris and/or aggregates. Indeed, data presented in this specification (FIGS. 1 and 2) show dramatic declines in CTC counts in several blood specimens stored at room temperature for 24 hours or longer, indicating substantial in vitro destruction of CTC after blood draw. While the losses of hematopoietic cells during storage are well known phenomena and the subject of above-cited patents by Streck Labs and by others, the occurrence of mechanical damage due to mixing or transport have to date not been recognized factors in the loss of CTC or rare cells. The formation of cellular debris and the interfering effects of accumulating debris and aggregates in the analysis of CTC or other rare cells have similarly been unrecognized to date. It appears to be most evident and problematic in highly sensitive enrichment assays requiring processing of relatively large blood volumes (5-50 mL), and subsequent microscopic detection or imaging of target cells after volume reduction (less than 1 mL).

Such debris are either not normally seen, or do not interfere in conventional non-enrichment assays, for example, by flow cytometry or in enrichment by density gradients methods.

In summary, all or some of the above-cited factors can and were found to contribute to in vitro debris and/or clump formation that have been observed to interfere with detection of CTC by enrichment procedures as disclosed in this invention. Stabilizer compositions and uses thereof, as disclosed in this invention, have been unexpectedly found to preserve CTC, dramatically improve the quality of blood specimens primarily by reducing formation of detectable cellular debris and aggregates, and to serve as stabilizers and fixatives in the enrichment processes. Most importantly, the discovery of effective stabilizers for minimizing in vitro damage to CTC and degradation in the quality of the blood specimens provide a more complete picture of the in vivo status of the CTC in a patient. The number of Intact CTC, damaged, or Suspect CTC, as well as the degree of damage to the CTC, as illustrated in FIG. 5, serve as diagnostically important indicators of the tumor burden, the proliferative potential of the tumor cells, and/or the effectiveness of therapy. In contrast, current art using non-stabilized samples superimposes unavoidable in vivo damage to CTC on avoidable in vitro storage and processing damage, and thus may yield erroneous information on CTC and tumor burdens in patients. Analysis of fragments and debris from damaged CTC is important to understand what is taking place in the patient. Therefore, methods and reagents for performing such analysis is described in a co-pending application entitled, “Analysis of circulating tumor cells, fragments, and debris.” That commonly-owned application is incorporated by reference herein.

The invention is illustrated by the following Examples herein, which are not intended to limit the scope of the invention, but rather provide cases for the general uses of the invention.

### EXAMPLE 1

### Stabilization of Circulating Tumor Cells in Blood

Cyto-Chex™, StabilCyte™ and TRANSfx™ are examples of three stabilizers that are commercially available and have shown utility in stabilizing blood cells in blood specimens for extended time periods. These stabilizers are optimized to maintain cell size (mainly by minimizing shrinking) and to preserve antigens on cell surfaces, primarily as determined by flow cytometry. The intended applications generally involve direct analyses and do not require extensive manipulation of the sample or enrichment of particular cell populations. In contrast, the circulating tumor cells, or other rare target cells, isolated and detected in this invention, comprise and are defined as pathological abnormal or rare cells present at very low frequencies, thus requiring substantial enrichment prior to detection.

CTC are often detectable in blood even after storage for 24 hours at room temperature or 2-8° C., but may become more fragile for reasons previously discussed. It was shown that any manipulation or enrichment methods could damage these fragile cells, thus resulting in potential cell losses and debris or aggregate formation during the process of isolation. FIGS. 1 and 2 illustrate the substantial losses of CTC in some specimens on storage for up to 24 hours.

In this Example, the effect of different stabilizers on recovery of CTC after enrichment from 24-hour-old specimens was examined. Blood samples from patients with advanced carcinomas were obtained and treated with stabilizers within 2 hours of blood draw as follows. Blood drawn from each patient into different tubes containing EDTA was pooled, and equal volumes were aliquotted into separate tubes. Various additives, consisting of Cyto-Chex™ stabilizer, StabilCyte™ stabilizer, and TRANSfix™ stabilizer, were added to the separate tubes at 30% Cyto-Chex™ stabilizer, 20% StabilCyte™ stabilizer, and 10% TRANSfix™ stabilizer, where these are percent of the blood volume. One tube was used as the control to which no buffer or stabilizer was added. The samples were then mixed and stored at room temperature for 24 hours. An equal volume of Immunicon System Buffer (PBS containing 0.5% BSA, 0.2% casein and 0.1% sodium azide) was added to each sample. After mixing, the samples were centrifuged at 800×g for 10 minutes to remove plasma. Immunicon AB Buffer (System Buffer containing streptavidin as a mediator of controlled reversible aggregation (techniques described in U.S. applications Ser. No. 09/351,515 and U.S. Pat. No. 09/702,188 incorporated by reference herein) was added to each tube to a final volume of 1.5× the initial blood volume. After mixing the samples, CA EpCAM ferrofluid (0.2 μm magnetic particles coupled to anti-EpCAM antibody and with desthiobiotin as a mediator of controlled reversible aggregation) was added to the samples to magnetically label CTC.

The samples were incubated with the CA EpCAM ferrofluid inside Immunicon quadrupole high-gradient magnetic separators (QMS 17; patented under U.S. Pat. Nos. 5,186,827 and 5,466,574 both incorporated by reference herein) for two 10 minute time periods with remixing outside the QMS17 magnets after each 10 minute time period, as described in U.S. application Ser. No. 09/240,939 incorporated by reference herein. After these magnetic incubations, the samples were magnetically separated for 20 minutes. The uncollected fractions were aspirated, and the tubes were removed from the QMS17. The magnetically collected fractions were resuspended in System Buffer and re-separated in the QMS17 for 10 minutes. The uncollected fractions were aspirated again and the collected cells were resuspended in 200 μl of Immuniperm™ (PBS containing 0.05% Saponin) to permeabilize the captured cells to allow intracellular staining.

The permeabilized samples were stained for 15 minutes with a cocktail consisting of several fluorescent markers: 20 μl anti-cytokeratin-PE, 20 μl anti-CD45-APC and 20 μl DAPI. Anti-cytokeratin stains epithelial cells and anti-CD45 stains leukocytes to differentiate any non-specifically stained leukocytes from target CTC. DAPI is used to identify all nucleated cells and to differentiate cells from non-nucleated cell debris. After washing out excess staining reagents by magnetic separation, the samples were resuspended in 320 μL Immunicon CellFix™ (System Buffer also containing 0.5% BSA, 10 mg/mL biotin and 25% Cyto-Chex™ stabilizer).

Each sample was transferred to an Immunicon CellSpotter® chamber (as described in U.S. application Ser. No. 10/074,900 incorporated by reference herein), a cuvet-like enclosure with an optically flat transparent upper viewing window designed to fit into a bipolar angled magnet assembly (as described in U.S. Pat. No. 6,136,182 incorporated by reference herein). Magnetic collection of the magnetically labeled target cells on the underside of the viewing window allows imaging by means of a fluorescence microscope. The sample chamber surface was automatically scanned with four different filters. The software collects and analyzes the images but presents only images that are positive for both cytokeratin-PE and DAPI as potential CTC candidates for subsequent re-viewing and classification. The classified Intact CTC, Suspect CTC, and “Not Assigned Events” (debris particles) were counted after confirming morphology and positive staining for cytokeratin-PE and DAPI, but negative staining for the pan-leukocyte marker, CD45-APC. Table 1 shows the effect of storing specimens from eight cancer patients in various stabilizers for 24 hours prior to analysis. The results are expressed as “Intact CTC”, “Suspect CTC” and “Not Assigned Events” events.

TABLE 1
Effect of stabilizers in clinical samples (after 24 hours at room temperature)

| | Intact CTC | | | | Suspect CTC | | | | Not Assigned Events | | | |
|---|---|---|---|---|---|---|---|---|---|---|---|---|
| Patient I.D. | N | T | S | C | N | T | S | C | N | T | S | C |
| D12 | 1 | 3 | 6 | 2 | 5 | 4 | 4 | 7 | 72 | 31 | 17 | 34 |
| (Breast cancer) | | | | | | | | | | | | |
| JM38 | 23 | 72 | 61 | 72 | 61 | 161 | 169 | 173 | 324 | 160 | 61 | 55 |
| (Prostate cancer) | 19 | 52 | 59 | 61 | 73 | 197 | 191 | 144 | 242 | 135 | 56 | 66 |
| JM33 | 14 | 20 | 23 | 26 | 25 | 29 | 27 | 33 | 357 | 76 | 93 | 68 |
| (Prostate cancer) | 27 | — | 25 | 28 | 22 | — | 32 | 25 | 360 | — | 98 | 76 |
| JM40 | 9 | 107 | 101 | 121 | 198 | 358 | 323 | 345 | 1034 | 1075 | 500 | 635 |
| (Prostate cancer) | | | | | | | | | | | | |
| D13 | 1 | 2 | 0 | 3 | 3 | 8 | 4 | 9 | 143 | 180 | 187 | 70 |
| (Breast cancer) | | | | | | | | | | | | |
| D9 | 58 | 106 | 81 | 89 | 46 | 41 | 42 | 38 | 421 | 105 | 107 | 135 |
| (Breast cancer) | | | | | | | | | | | | |
| JDS002 | 0 | 1 | 0 | 0 | 1 | 2 | 1 | 0 | 82 | 48 | 92 | 82 |
| (Breast cancer) | | | | | | | | | | | | |
| JDS009 | 0 | 0 | 0 | 0 | 0 | 2 | 0 | 0 | 47 | 44 | 12 | 32 |
| (Breast cancer) | | | | | | | | | | | | |

N = No stabilizer
T = TRANSfix ™ stabilizer
S = StabilCyte ™ stabilizer
C = Cyto-Chex ™ stabilizer

Higher numbers of Intact and Suspect CTC were detected in most of the patient samples after 24-hour storage of these blood samples with all three stabilizers when compared to blood samples with no added stabilizer. It may be possible that substantial conversions of Intact CTC to Suspect CTC had occurred in unstabilized specimens, or that the cells were lost. Statistical analysis of these data show significant differences in numbers of total CTC detected between stabilizers and no stabilizers (p-value=0.02). This difference was seen with both breast and prostate cancer samples. However, there was no difference between the three stabilizers tested (p-values=0.18). The higher numbers of Intact and Suspect CTC detected in the presence of stabilizers are not believed to be artifacts due to non-specific staining, as this effect was not observed in 24-hour old samples from normal donors. These data clearly show that addition of stabilizers to blood samples from cancer patients preserve circulating tumor cells during storage and in the sample processing steps, thus permitting more accurate classification of in vivo levels of Intact CTC and Suspect CTC.

### EXAMPLE 2

### Preservation of Sample Quality for Analysis

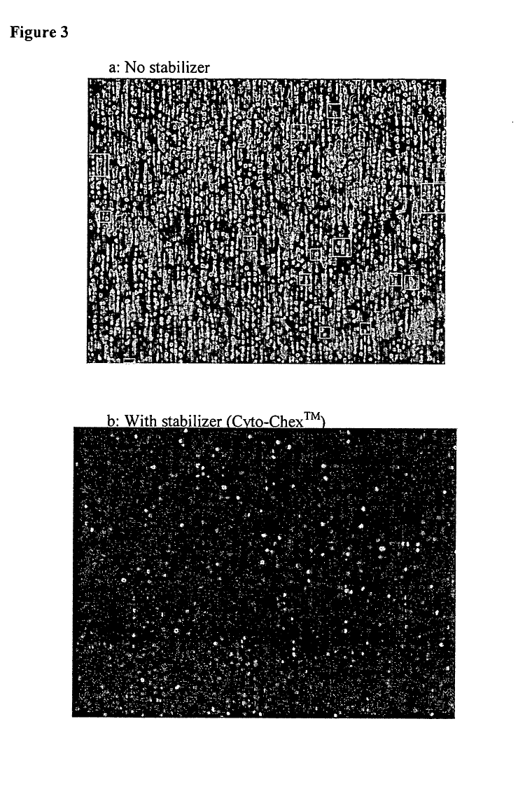

CTC are present in blood at a low frequency and require large sample volumes and efficient enrichment methods for detection. Enrichment methods for CTC involve several wash steps, including magnetic separation methods that can damage cells and create debris and clumps due to DNA leakage from cells. However, it was found unexpectedly, and rather surprisingly, that mild end-over-end mixing of blood tubes on a nutator, as routinely done in most hematology labs to keep the blood cells suspended, causes significant formation of cellular debris that can interfere with detection and enumeration of CTC. Furthermore, it was observed that incomplete filling of the blood draw or assay tubes further aggravated this mixing damage, but no damage was observed in stationary tubes. As shown in FIG. 3, any mixing of patient samples during shipping or other mechanical stress was also shown to unexpectedly increase interfering debris. Table II shows the effect of Cyto-Chex™ stabilizer on the recovery of CTC after overnight shipment of the blood samples. The detection of CTC requires large sample volumes as well as special skills and equipment that are not routinely available in every clinical laboratory. As a result, those blood samples need to be shipped to a central lab for processing. As mentioned earlier, CTC are delicate and any mechanical mixing or shaking might damage the cells during shipping. Our data show that it is absolutely essential to add a stabilizing agent as disclosed in this invention to preserve both CTC and sample quality prior to transport, storage, and processing of blood specimens.

These adverse effects on CTC and sample quality may have escaped detection to date, since such stressed samples may still be satisfactory for most laboratory or clinical analyses. For example, immunophenotyping by flow cytometry do not require any enrichment methods and where the presence of debris apparently does not interfere with analysis. However, our findings suggest that it is critically important to preserve blood sample quality when assaying enriched rare target cells by microscopic or other optical methods.

### EXAMPLE 3

### Effect of Cyto-Chex# Stabilizer on Sample Quality of Normal Specimens Mixed for 2-3 Hours

Staining of the cell nucleus with DAPI normally is detectable inside a permeabilized live or dead cell, if the nucleus is intact. DNA staining may also be detectable in cell fragments or cellular debris, such as stainable aggregates outside the cell if DNA has leaked out. Staining with DAPI and examination under a fluorescence microscope can thus readily check the sample quality.

Three tubes of blood were drawn into 10 mL EDTA anti-coagulated tubes from a normal donor. Cyto-Chex™ stabilizer was added to one blood tube without removal of the cap plug by using the CellStabilize™ injection device as follows. The blood tube was placed in the calibrated device with graduations that allows estimating the blood volume to permit addition of the proper amount of stabilizer to the desired final concentration. One needle (27G, ½ inch) was inserted through the stopper of the tube as a vent and the required volume of stabilizer was injected into the tube using a second needle (20G, 1½ inch) and a syringe. The specimen tube was removed from the device and mixed by several inversions of the tube. Two blood specimens, one without stabilizer and another tube with 30% Cyto-Chex™ stabilizer were mixed for 2-3 hours on a nutator mixer to mimic shipping conditions. The third blood tube was not mixed for use as a control without stabilizer. After 2-3 hours, 7.5 mL of blood was transferred from each “no stabilizer” tube while 9.75 mL of blood was transferred from the “Cyto-Chex™ stabilizer” tube to separate 15 ml polypropylene centrifuge tubes for processing as described in Example 1.

The samples were transferred to Immunicon CellSpotter® chambers for CellSpotter® analysis using fluorescent imaging microscopy. The samples were scanned in four different filters for target cells that had been magnetically aligned on the underside of the viewing window of the chamber. The images from different filters were stored. The sample quality was determined from the number of detectable DAPI stained events (Intact CTC, Suspect CTC, and stainable “Not Assigned Events” or debris) compared to the number of detectable nucleated CTC and non-target cells. For optimal sample quality, stainable cellular debris and aggregates must be kept small to permit accurate detection and enumeration of Intact CTC and Suspect CTC.

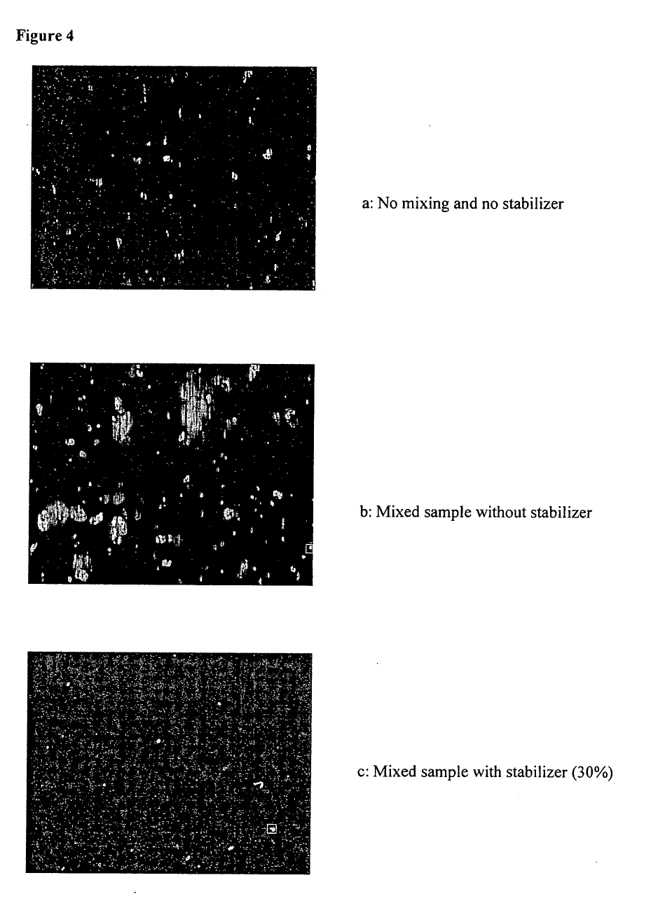

The images in FIG. 3 show the damaging effect of transporting a blood tube without a stabilizer (FIG. 3a) compared to a tube with 30% Cyto-Chex stabilizer added (FIG. 3b). Similarly, FIGS. 4a, 4b, and 4c show the staining of cell nuclei with DAPI. There were numerous DAPI stainable cell clumps and DNA debris when samples were mixed without any stabilizer present (FIG. 4b) as compared to the control samples with no stabilizer or mixing (FIG. 4a) and samples with Cyto-Chex™ stabilizer plus mixing (FIG. 4c). These data unexpectedly show that mixing of blood samples damages not only the fragile CTC, but also normal hematopoietic cells, thereby generating debris and aggregates that can be detected after enrichment. Surprisingly, addition of stabilizers to blood samples before mixing the samples was found to minimize both cell damage and to preserve sample quality.

### EXAMPLE 4

### Effect of Cyto-Chex™ Stabilizer on Circulating Tumor Cells During Shipping

In this Example, two tubes of EDTA anti-coagulated blood were drawn from 31 cancer patients (3 were repeat patients received two separate times) with advanced stage disease. Cyto-Chex™ stabilizer was directly injected into one set of tubes using venting with the CellStabilize™ device described in Example 3, such that final volume of Cyto-Chex™ was 30% the total volume. The second set of tubes was untreated controls. Both sample sets were then shipped to Immunicon by overnight service with no ice packs. The day-old samples were processed by enrichment of the CTC and detection by CellSpotter™ analysis. The procedure used in this Example to enrich and detect the CTC is similar to the procedure used in Example 1. The results on blood samples with and without added Cyto-Chex™ stabilizer are shown in Table 2.

TABLE 2

| | | | | | | Not Assigned | |
|---|---|---|---|---|---|---|---|
| | | Intact CTC | | Suspect CTC | | Events | |
| | | With- | | With- | | With- | |
| Patient ID # | Cancer Type | out | With | out | With | out | With |
| 20370 (1) | Breast | 0 | 1 | 1 | 4 | 44 | 21 |
| 7493 * (2) | Breast | 9 | 16 | 3 | 4 | 35 | 20 |
| 23435 (3) | Breast | 0 | 0 | 0 | 0 | 55 | 40 |
| 24948 (4) | Lung | 2 | 3 | 2 | 8 | 292 | 307 |
| 10520 (5) | Breast | 0 | 7 | 0 | 12 | 1939 | 104 |
| 26858 (6) | Uterus | 0 | 1 | 0 | 0 | 53 | 24 |
| 25000 (7) | Prostate | 0 | 2 | 0 | 4 | 801 | 242 |
| 23549 (8) | Lung | 1 | 1 | 11 | 1 | 3194 | 135 |
| 25167 (9) | Breast | 0 | 0 | 0 | 0 | 31 | 28 |
| 26041 (10) | Colon | 0 | 0 | 0 | 4 | 649 | 120 |
| 24164 (11) | Endometrial | 0 | 0 | 0 | 2 | 628 | 86 |
| 20942 (12) | Breast | 0 | 0 | 1 | 0 | 201 | 115 |
| 28217 (13) | Breast | 1 | 4 | 0 | 7 | 52 | 33 |
| 19710 (14) | Colon | 0 | 0 | 0 | 0 | 110 | 27 |
| 19218 (15) | Lung | 0 | 0 | 1 | 0 | 53 | 25 |
| 28162 (16) | Rectum | 0 | 0 | 1 | 0 | 1544 | 79 |
| 23530 (17) | Prostate | 21 | 106 | 26 | 42 | 142 | 179 |
| 24948 (18) | Lung | 0 | 6 | 3 | 7 | 283 | 46 |
| 28287 (19) | Lung | 0 | 1 | 0 | 1 | 155 | 32 |
| 27851 * (20) | Renal | 0 | 0 | 0 | 1 | 12 | 13 |
| 28186 * (21) | Uterus | 0 | 0 | 1 | 0 | 33 | 21 |
| 21515 * (22) | Breast | 0 | 0 | 0 | 0 | 17 | 20 |
| 25084 * (23) | Ovary | 0 | 0 | 0 | 0 | 17 | 63 |
| 25000 * (24) | Prostate | 0 | 6 | 2 | 1 | 25 | 63 |
| 25254 * (25) | Lung | 0 | 0 | 0 | 0 | 20 | 24 |
| 22488 (26) | Breast | 2 | 0 | 2 | 3 | 19 | 28 |
| 21688 (27) | Breast | 0 | 2 | 3 | 3 | 36 | 91 |
| 16925 (28) | Breast | 2 | 10 | 3 | 3 | 76 | 64 |
| 28327 * (29) | Bladder | 62 | 285 | 105 | 72 | 139 | 113 |
| 19218 (30) | Lung | 0 | 0 | 0 | 0 | 19 | 41 |
| 13517 (31) | Prostate | 8 | 16 | 7 | 5 | 270 | 39 |

Without - no stabilizer added
With - 30% (final volume) Cyto-Chex ™ added
* = 5 ml specimen, all others 7.5 ml

Any sample that showed more than one Intact CTC was considered as positive. Applying the Wilcoxon signed rank test to the data sets without and with stabilizer gave the following p-values:
Intact CTC, p<0.0004 (highly significant),
Suspect CTC, p<0.155 (not significant), and
Not Assigned Events, p<0.0327 (significant).

Most specimens became positive or showed increases in Intact CTC when Cyto-Chex™ stabilizer was added. The cases where more Intact CTC or Suspect CTC were detected in the non-stabilized specimens are probably artifactual and the result of difficulty in differentiating stainable debris and aggregates without characteristic cell morphology, classified as “Not Assigned Events” from Intact or Suspect CTC. About three times more Intact CTC, on the average, were detected with stabilized specimens compared to non-stabilized specimens. These data highlight the unexpected preservative effect of whole blood stabilizers in preserving both tumor cells and specimen quality.

### EXAMPLE 5

### 24 vs. 72 Hour Stabilization

In the shipping study (Example 4), it was shown that stabilization preserves cells in samples processed 24 hours after the blood was drawn. However, it would also be desirable to have cells stable for at least 72 hours. In this Example, the blood samples were processed 24 and 72 hours after the blood draw and analyzed as follows: A minimum of two 10 mL EDTA Vacutainer® tubes of blood (˜8 mL of blood per tube) were obtained from metastatic cancer patients. Within 15 minutes of the blood draw, an appropriate amount of CytoChex™ was manually added to each of the EDTA tubes to yield 30% CytoChex™ by volume using the CellStabilize™ injection device as described in Example 3.

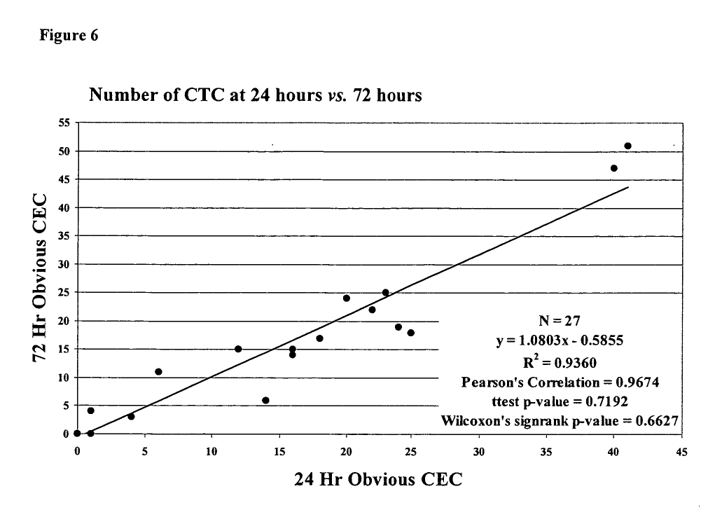

Upon receipt of the blood specimens, the blood was pooled, and 7.5 mL of stabilized blood was tested at 24 hours and/or 72 hours using the same assay and analyzed using the same CellSpotter® system analysis as described in Example 1. The CTC were compared at these two time points. At 90% power, to detect an expected correlation coefficient of 0.95 with a two-sided p-value of 0.01, a total of 8 specimens with paired values are required. In the 24 hours vs. 72 hours comparison, there were a total of 28 specimens with paired values, however one sample had extremely high CTC, and is not present in FIG. 6.

FIG. 6 shows the plot of 27 specimens with obvious CTC results at 24 hours vs. 72 hours. The regression equation, r-squared value, correlation coefficient, and the t-test p-value are shown on the graph. The r-squared value and Pearson's correlation coefficient show that the CTC counts after 24 hours are highly correlated with those at 72 hours, and the t-test shows that the means of the two counts are not statistically significantly different (24 hr=11, 72 hr=11). The slope of the line (1.0803) indicates approximately the same number of obvious CTC after 24 hours or 72 hours of incubation with the blood stabilizer. Wilcoxon's non-parametric sign-rank test further showed that the two CTC counts at these two time points are not significantly different (p=0.9105). The results show that there is no loss of CTC after 72 hours of incubation with the stabilizer, and that the counts at 72 hours are almost identical to those at 24 hours.

### EXAMPLE 6

### Modes of Addition of Stabilizers

As mentioned earlier, CTC in the blood exist in different forms (such as Intact, damaged, etc.) and in general they are fragile. The stabilizer needs to be added as soon as blood is drawn into the tube to prevent further cellular damage in the tube. However, it is a difficult process to control the time of addition, which depends upon the place of blood draw. Some sites where blood is drawn may not necessarily have a stabilizing agent, and therefore need to send the samples to another site to add a stabilizer. This creates variable time of stabilizer addition. Even in these sites, the time of stabilizer addition will depend upon the number of tubes drawn. The phlebotomist who draws blood may not add a stabilizer, but would transfer samples to other technicians for stabilizer addition.

Therefore, the preferred stabilizers may be used as concentrated liquids or in diluted form as solutions in water or buffers to minimize transient exposure to hyperosmotic conditions and potential damage to cells. The liquid or solid stabilizers are preferably added to the blood draw container prior to blood draw or, less optimally, immediately thereafter to gain the maximum protective effect from damage due to turbulence during blood draw.

Pre-filled blood collection tubes, e.g. 10 mL Vacutainer® tubes (Becton Dickinson, Franklin Lakes, N.J.) or Vacuette® tubes (from Greiner America, Lake Mary, Fla.), are commercially available, pre-filled with about 2 mL liquid reagents or with particulate coating materials prior to evacuation. Alternative means for manufacturing pre-filled tubes include conventional in situ lyophilization of frozen liquids. Another patented technique utilizes discrete freeze-dried beads, so-called LyoSpheres (U.S. Pat. No. 6,106,836; Akzo Nobel Nev., Netherlands), and Biolyph, (Minneapolis, Minn.), which are prepared by dropping liquid droplets of up to 250 μL into liquid nitrogen followed by conventional lyophilization of the frozen beads inside the container to form discrete rigid beads.

In summary, Examples 1-6 show that the addition of stabilizers to blood samples preserves sample quality as well as CTC in the enrichment assays utilized in this invention. It is to be appreciated that the optimum amounts of stabilizers will vary, and depend on the type and physical state of the stabilizer, ranging from liquids to lyophilized pellets to powders as recited in Example 6. For example, although it has been found that the concentrations of Cyto-Chex™ stabilizer can range from 1-50%, preferably about 5-40% and most preferably about 10-30%, and from 0.1-30%, preferably about 1-25% and most preferably about 1-10% for TRANSfix™ stabilizer, it is to be appreciated that the concentrations or amounts for other types of stabilizers may differ from the specified ranges and must be optimized for each stabilizer and application. Ideally, anti-coagulating agents and said stabilizing agents would be present in volumes of about 0.1-50%, more preferably about 0.3-30%, and most preferably about 0.3-5% of the total volume of the biological specimen, while maintaining the same effective concentration of the stabilizing agent. More importantly, less dilution is more desirable.

The improvements provided by the present invention will be readily apparent to one skilled in the art by reference to the above-described US patents, and the preferred embodiments described herein. One particularly advantageous aspect of the present invention is that it discloses stabilizers for preserving rare target cells and CTC in whole blood during storage and during enrichment processes. Further improvements provided by the invention result from the addition of the stabilizers prior to or shortly after blood draw, and the utilization of the stabilizers in both liquid and solid forms. The improvements provided by the invention have been described and exemplified in terms of preferred embodiments in the above-cited Examples 1-6.

The invention further improves the analysis of Intact CTC, Suspect CTC, and Not Assigned Events, the latter consisting of debris and/or aggregates present at blood draw or formed during processing. The present invention is the result of an extensive research project that was undertaken in the development of the improved compositions and methods of the present invention, which particularly explored the compositions and modes for stabilizing rare cells, particularly of CTC. The preferred embodiments of the invention, which incorporate these improvements, as herein disclosed, are also believed to enable the invention to be employed in fields and applications additional to cancer diagnosis. It will be apparent to those skilled in the art that the improved diagnostic modes of the invention are not to be limited by the foregoing descriptions of preferred embodiments. Finally, while certain embodiments presented above provide detailed descriptions, the following claims are not limited in scope by the detailed descriptions. Indeed, various modifications may be made thereto without departing from the spirit of the following claims.

1. A composition for preserving biological specimens consisting of:
a. an anti-coagulating agent, and
b. a stabilizing agent.

2. The composition of claim 1, wherein said anti-coagulating agent is a chelating agent.

3. The composition of claim 2, wherein said anti-coagulating agent is selected from the group consisting of: ethylenediamine tetraacetic acid (EDTA), diethylenetriamine pentaacetic acid (DTPA), 1,2-diaminocyclohexane tetraacetic acid (DCTA), and ethylenebis(oxyethylenenitrilo) tetraacetic acid (EGTA).

4. The composition of claim 1, wherein said anti-coagulating agent is a complexing agent.

5. The composition of claim 4, wherein said anti-coagulating agent is selected from the group consisting of heparin and citrate.

6. The composition of claim 1, wherein said stabilizing agent is a formaldehyde donor.

7. The composition of claim 6, wherein said formaldehyde donor is selected from the group consisting of: methylol or hydroxymethyl derivatives of amines or amides, diazolinidinyl urea, imidazolidinyl urea, methenamine, and paraformaldehyde.

8. The composition of claim 1, wherein said stabilizing agent is an aldehyde.

9. The composition of claim 8, wherein said aldehyde is selected from the group consisting of: formaldehyde, glutaraldehyde, and glyoxal.

10. The composition of claim 1, wherein said stabilizing agent is a formaldehyde donor or an aldehyde combined with at least one heavy metal element.

11. The composition of claim 10, wherein said heavy metal element is selected from the group consisting of: chromium, manganese, and zinc.

12. The composition of claim 1, wherein an additional stabilizing agent is polyethylene glycol.

13. The composition of claim 12, wherein the molecular weight of said polyethylene glycol is in the range of about 1000 to about 35000.

14. The composition of claim 12, wherein the molecular weight of said polyethylene glycol is in the range of about 5000 to about 20000.

15. The composition of claim 12, wherein the molecular weight of said polyethylene glycol is in the range of about 8000 to about 20000.

16. A composition for preserving blood samples suspected to contain circulating tumor cells consisting of:
a. an anti-coagulating agent, and
b. a stabilizing agent.

17. The composition of claim 16, wherein said anti-coagulating agent is a chelating agent.

18. The composition of claim 17, wherein said anti-coagulating agent is selected from the group consisting of: ethylenediamine tetraacetic acid (EDTA), diethylenetriamine pentaacetic acid (DTPA), 1,2-diaminocyclohexane tetraacetic acid (DCTA), and ethylenebis(oxyethylenenitrilo) tetraacetic acid (EGTA).

19. The composition of claim 16, wherein said anti-coagulating agent is a complexing agent.

20. The composition of claim 19, wherein said anti-coagulating agent is selected from the group consisting of heparin and citrate.

21. The composition of claim 16, wherein said stabilizing agent is a formaldehyde donor.

22. The composition of claim 21, wherein said formaldehyde donor is selected from the group consisting of: methylol or hydroxymethyl derivatives of amines or amides, diazolinidinyl urea, imidazolidinyl urea, methenamine, and paraformaldehyde.

23. The composition of claim 16, wherein said stabilizing agent is an aldehyde.

24. The composition of claim 23, wherein said aldehyde is selected from the group consisting of: formaldehyde, glutaraldehyde, and glyoxal.

25. The composition of claim 16, wherein said stabilizing agent is a formaldehyde donor or an aldehyde combined with at least one heavy metal element.

26. The composition of claim 25, wherein said heavy metal element is selected from the group consisting of: chromium, manganese, and zinc.

27. The composition of claim 16, wherein an additional stabilizing agent is polyethylene glycol.

28. The composition of claim 27, wherein the molecular weight of said polyethylene glycol is in the range of about 1000 to about 35000.

29. The composition of claim 27, wherein the molecular weight of said polyethylene glycol is in the range of about 5000 to about 20000.

30. The composition of claim 27, wherein the molecular weight of said polyethylene glycol is in the range of about 8000 to about 20000.

31. A stabilized cell composition consisting of:
a. a biological specimen,
b. an anti-coagulating agent, and
c. a stabilizing agent.

32. The stabilized cell composition of claim 31, wherein said biological specimen is a fraction of blood suspected to contain circulating tumor cells.

33. The stabilized cell composition of claim 32, wherein said circulating tumor cells have been stabilized by said stabilizing agent.

34. The stabilized cell composition of claim 31, wherein said anti-coagulating agent is a chelating agent.

35. The stabilized cell composition of claim 34, wherein said anti-coagulating agent is selected from the group consisting of: ethylenediamine tetraacetic acid (EDTA), diethylenetriamine pentaacetic acid (DTPA), 1,2-diaminocyclohexane tetraacetic acid (DCTA), and ethylenebis(oxyethylenenitrilo) tetraacetic acid (EGTA).

36. The stabilized cell composition of claim 31, wherein said anti-coagulating agent is a complexing agent.

37. The stabilized cell composition of claim 36, wherein said anti-coagulating agent is selected from the group consisting of heparin and citrate.

38. The stabilized cell composition of claim 31, wherein said stabilizing agent is a formaldehyde donor.

39. The stabilized cell composition of claim 38, wherein said formaldehyde donor is selected from the group consisting of: methylol or hydroxymethyl derivatives of amines or amides, diazolinidinyl urea, imidazolidinyl urea, methenamine, and paraformaldehyde.

40. The stabilized cell composition of claim 31, wherein said stabilizing agent is an aldehyde.

41. The stabilized cell composition of claim 40, wherein said aldehyde is selected from the group consisting of: formaldehyde, glutaraldehyde and glyoxal.

42. The stabilized cell composition of claim 31, wherein said stabilizing agent is formaldehyde donor or an aldehyde combined with at least one heavy metal element.

43. The stabilized cell composition of claim 42, wherein said heavy metal element is selected from the group consisting of: chromium, manganese, and zinc.

44. The stabilized cell composition of claim 31, wherein an additional stabilizing agent is polyethylene glycol.

45. The stabilized cell composition of claim 44, wherein the molecular weight of said polyethylene glycol is in the range of about 1000 to about 35000.

46. The stabilized cell composition of claim 44, wherein the molecular weight of said polyethylene glycol is in the range of about 5000 to about 20000.

47. The stabilized cell composition of claim 44, wherein the molecular weight of said polyethylene glycol is in the range of about 8000 to about 20000.

48. The stabilized cell composition of claim 31, wherein said anti-coagulating agents and said stabilizing agents are present in volumes of about 0.1 to about 50% of the total volume of said biological specimen.

49. The stabilized cell composition of claim 47, wherein said volumes are in the range of about 0.3 to about 30% of the total volume of said biological specimen.

50. The stabilized cell composition of claim 47, wherein said volumes are in the range of about 0.3 to about 5% of the total volume of said biological specimen.

51. A method for preserving biological specimens consisting of:
a. obtaining a biological specimen that contains cells, and
b. contacting said biological specimen with a stabilizing agent capable of stabilizing said cells.

52. The method of claim 51, wherein said stabilizing agent is a formaldehyde donor.

53. The method of claim 52, wherein said formaldehyde donor is selected from the group consisting of: methylol or hydroxymethyl derivatives of amines or amides, diazolinidinyl urea, imidazolidinyl urea, methenamine, and paraformaldehyde.

54. The method of claim 51, wherein said stabilizing agent is an aldehyde.

55. The method of claim 54, wherein said aldehyde is selected from: formaldehyde, glutaraldehyde, and glyoxal.

56. The method of claim 51, wherein said stabilizing agent is formaldehyde donor or an aldehyde combined with at least one heavy metal element.

57. The method of claim 56, wherein said heavy metal element is selected from the group consisting of: chromium, manganese, and zinc.

58. The method of claim 51, wherein an additional stabilizing agent is polyethylene glycol.

59. The method of claim 58, wherein the molecular weight of said polyethylene glycol is in the range of about 1000 to about 35000.

60. The method of claim 58, wherein the molecular weight of said polyethylene glycol is in the range of about 5000 to about 20000.

61. The method of claim 58, wherein the molecular weight of said polyethylene glycol is in the range of about 8000 to about 20000.

62. The method of claim 51, wherein said specimen is further contacted with an anti-coagulating agent.

63. The method of claim 62, wherein said anti-coagulating agent is a chelating agent.

64. The method of claim 63, wherein said anti-coagulating agent is selected from the group consisting of: ethylenediamine tetraacetic acid (EDTA), diethylenetriamine pentaacetic acid (DTPA), 1,2-diaminocyclohexane tetraacetic acid (DCTA), and ethylenebis(oxyethylenenitrilo) tetraacetic acid (EGTA).

65. The method of claim 62, wherein said anti-coagulating agent is a complexing agent.

66. The method of claim 65, wherein said anti-coagulating agent is selected from the group consisting of heparin and citrate.

67. The method of claim 62, wherein said anti-coagulating agent and said stabilizing agent are combined before contacting said biological specimen.

68. The method of claim 67, wherein said anti-coagulating agent is a chelating agent.

69. The method of claim 68, wherein said anti-coagulating agent is selected from the group consisting of: ethylenediamine tetraacetic acid (EDTA), diethylenetriamine pentaacetic acid (DTPA), 1,2-diaminocyclohexane tetraacetic acid (DCTA), and ethylenebis(oxyethylenenitrilo) tetraacetic acid (EGTA).

70. The method of claim 67, wherein said anti-coagulating agent is a complexing agent.

71. The method of claim 70, wherein said anti-coagulating agent is selected from the group consisting of heparin and citrate.

72. The method of claim 67, wherein said anti-coagulating agents and said stabilizing agents are present in volumes of about 0.1 to about 50% of the total volume of said biological specimen.

73. The method of claim 72, wherein said volumes are in the range of about 0.3 to about 30% of the total volume of said biological specimen.

74. The method of claim 72, wherein said volumes are in the range of about 0.3 to 5% of the total volume of said biological specimen.

75. A method for preserving blood samples suspected to contain circulating tumor cells consisting of:
a. obtaining a biological specimen that contains cells, and
b. contacting said biological specimen with a stabilizing agent capable of stabilizing said cells.

76. The method of claim 75, wherein said stabilizing agent is a formaldehyde donor.

77. The method of claim 76, wherein said formaldehyde donor is selected from the group consisting of: methylol or hydroxymethyl derivatives of amines or amides, diazolinidinyl urea, imidazolidinyl urea, methenamine, and paraformaldehyde.

78. The method of claim 75, wherein said stabilizing agent is an aldehyde.

79. The method of claim 78, wherein said aldehyde is selected from the group consisting of: formaldehyde, glutaraldehyde, and glyoxal.

80. The method of claim 75, wherein said stabilizing agent is formaldehyde donor or an aldehyde combined with at least one heavy metal element.

81. The method of claim 80, wherein said heavy metal element is selected from the group consisting of: chromium, manganese, and zinc.

82. The method of claim 75, wherein an additional stabilizing agent is polyethylene glycol.

83. The method of claim 82, wherein the molecular weight of said polyethylene glycol is in the range of about 1000 to about 35000.

84. The method of claim 82, wherein the molecular weight of said polyethylene glycol is in the range of about 5000 to about 20000.

85. The composition of claim 82, wherein the molecular weight of said polyethylene glycol is in the range of about 8000 to about 2000.

86. The method of claim 75, wherein said specimen is further contacted with an anti-coagulating agent.

87. The method of claim 86, wherein said anti-coagulating agent is a chelating agent.

88. The method of claim 87, wherein said anti-coagulating agent is selected from the group consisting of: ethylenediamine tetraacetic acid (EDTA), diethylenetriamine pentaacetic acid (DTPA), 1,2-diaminocyclohexane tetraacetic acid (DCTA), and ethylenebis(oxyethylenenitrilo) tetraacetic acid (EGTA).

89. The method of claim 86, wherein said anti-coagulating agent is a complexing agent.

90. The method of claim 89, wherein said anti-coagulating agent is selected from the group consisting of heparin and citrate.

91. The method of claim 86, wherein said anti-coagulating agent and said stabilizing agent are combined before contacting said biological specimen.

92. The method of claim 91, wherein said anti-coagulating agent is a chelating agent.

93. The method of claim 92, wherein said anti-coagulating agent is selected from the group consisting of: ethylenediamine tetraacetic acid (EDTA), diethylenetriamine pentaacetic acid (DTPA), 1,2-diaminocyclohexane tetraacetic acid (DCTA), and ethylenebis(oxyethylenenitrilo) tetraacetic acid (EGTA).

94. The method of claim 91, wherein said anti-coagulating agent is a complexing agent.

95. The method of claim 93, wherein said anti-coagulating agent is selected from the group consisting of heparin and citrate.

96. The method of claim 86, wherein said anti-coagulating agents and said stabilizing agents are present in volumes of about 0.1 to about 50% of the total volume of said biological specimen.

97. The method of claim 96, wherein said volumes are in the range of about 0.3 to about 30% of the total volume of said biological specimen.

98. The method of claim 96, wherein said volumes are in the range of about 0.3 to about 5% of the total volume of said biological specimen.

99. An apparatus for preserving biological specimens consisting of an evacuated blood draw tube, said tube containing:
a. an anti-coagulating agent, and
b. a stabilizing agent.

100. The apparatus of claim 99, wherein said anti-coagulating agent is a chelating agent.

101. The apparatus of claim 100, wherein said anti-coagulating agent is selected from the group consisting of: ethylenediamine tetraacetic acid (EDTA), diethylenetriamine pentaacetic acid (DTPA), 1,2-diaminocyclohexane tetraacetic acid (DCTA), and ethylenebis(oxyethylenenitrilo) tetraacetic acid (EGTA).

102. The apparatus of claim 99, wherein said anti-coagulating agent is a complexing agent.

103. The apparatus of claim 102, wherein said anti-coagulating agent is selected from the group consisting of heparin and citrate.

104. The apparatus of claim 99, wherein said stabilizing agent is a formaldehyde donor.

105. The apparatus of claim 104, wherein said formaldehyde donor is selected from the group consisting of: methylol or hydroxymethyl derivatives of amines or amides, diazolinidinyl urea, imidazolidinyl urea, methenamine, and paraformaldehyde.

106. The apparatus of claim 99, wherein said stabilizing agent is an aldehyde.

107. The apparatus of claim 106, wherein said aldehyde is selected from the group consisting of: formaldehyde, glutaraldehyde, and glyoxal.

108. The apparatus of claim 99, wherein said stabilizing agent is formaldehyde donor or an aldehyde combined with at least one heavy metal element.

109. The apparatus of claim 108, wherein said heavy metal element is selected from the group consisting of: chromium, manganese, and zinc.

110. The apparatus of claim 99, wherein said stabilizing agent has been lyophilized.

111. The apparatus of claim 99 wherein an additional stabilizing agent is polyethylene glycol.

112. The apparatus of claim 111, wherein the molecular weight of said polyethylene glycol is in the range of about 1000 to about 35000.

113. The apparatus of claim 112, wherein the molecular weight of said polyethylene glycol is in the range of about 5000 to about 20000.

114. The apparatus of claim 112, wherein the molecular weight of said polyethylene glycol is in the range of about 8000 to about 20000.

115. The apparatus of claim 111, wherein said additional stabilizing agent has been lyophilized.

116. The apparatus of claim 99, wherein said anti-coagulating agents and said stabilizing agents are present in volumes of about 0.1 to about 50% of the total volume of said draw tube.

117. The apparatus of claim 116, wherein said volumes are in the range of about 0.3 to about 30% of the total volume of said draw tube.

118. The apparatus of claim 116, wherein said volumes are in the range of about 0.3 to about 5% of the total volume of said draw tube.

119. An apparatus for preserving blood samples suspected to contain circulating tumor cells consisting of an evacuated blood draw tube containing:
a. an anti-coagulating agent, and
b. a stabilizing agent.

120. The apparatus of claim 119, wherein said anti-coagulating agent is a chelating agent.

121. The apparatus of claim 120, wherein said anti-coagulating agent is selected from the group consisting of: ethylenediamine tetraacetic acid (EDTA), diethylenetriamine pentaacetic acid (DTPA), 1,2-diaminocyclohexane tetraacetic acid (DCTA), and ethylenebis(oxyethylenenitrilo) tetraacetic acid (EGTA).

122. The apparatus of claim 119, wherein said anti-coagulating agent is a complexing agent.

123. The apparatus of claim 122, wherein said anti-coagulating agent is selected from the group consisting of heparin and citrate.

124. The apparatus of claim 119, wherein said stabilizing agent is a formaldehyde donor.

125. The apparatus of claim 123, wherein said formaldehyde donor is selected from the group consisting of: methylol or hydroxymethyl derivatives of amines or amides, diazolinidinyl urea, imidazolidinyl urea, methenamine, and paraformaldehyde.

126. The apparatus of claim 119, wherein said stabilizing agent is an aldehyde.

127. The apparatus of claim 126, wherein said aldehyde is selected from the group consisting of: formaldehyde, glutaraldehyde, and glyoxal.

128. The apparatus of claim 119, wherein said stabilizing agent is formaldehyde donor or an aldehyde combined with at least one heavy metal element.

129. The apparatus of claim 128, wherein said heavy metal element is selected from the group consisting of: chromium, manganese, and zinc.

130. The apparatus of claim 119, wherein said stabilizing agent has been lyophilized.

131. The apparatus of claim 119, wherein an additional stabilizing agent is polyethylene glycol.

132. The apparatus of claim 131, wherein the molecular weight of said polyethylene glycol is in the range of about 1000 to about 35000.

133. The apparatus of claim 131, wherein the molecular weight of said polyethylene glycol is in the range of about 5000 to about 20000.

134. The apparatus of claim 131, wherein the molecular weight of said polyethylene glycol is in the range of about 8000 to about 20000.

135. The apparatus of claim 131, wherein said additional stabilizing agent has been lyophilized.

136. The apparatus of claim 119, wherein said anti-coagulating agents and said stabilizing agents are present in volumes of about 0.1 to about 50% of the total volume of said draw tube.

137. The apparatus of claim 136, wherein said volumes are in the range of about 0.3 to about 30% of the total volume of said draw tube.

138. The apparatus of claim 136, wherein said volumes are in the range of about 0.3 to about 5% of the total volume of said draw tube.

139. The composition of claim 1, wherein an additional stabilizing agent is polyethylene glycol at a concentration of about 0.1% to about 5%, preferably about 0.1% to about 1%, and most preferably about 0.1% to about 0.5% of the specimen volume.

140. The composition of claim 16, wherein an additional stabilizing agent is polyethylene glycol at a concentration of about 0.1% to about 5%, preferably about 0.1% to about 1%, and most preferably about 0.1% to about 0.5% of the specimen volume.

141. The stabilized cell composition of claim 31, wherein an additional stabilizing agent is polyethylene glycol at a concentration of about 0.1% to about 5%, preferably about 0.1% to about 1%, and most preferably about 0.1% to about 0.5% of the specimen volume.

142. The method of claim 51, wherein an additional stabilizing agent is polyethylene glycol at a concentration of about 0.1% to about 5%, preferably about 0.1% to about 1%, and most preferably about 0.1% to about 0.5% of the specimen volume.

143. The method of claim 75, wherein an additional stabilizing agent is polyethylene glycol at a concentration of about 0.1% to about 5%, preferably about 0.1% to about 1%, and most preferably about 0.1% to about 0.5% of the specimen volume.

144. The apparatus of claim 99, wherein an additional stabilizing agent is polyethylene glycol at a concentration of about 0.1% to about 5%, preferably about 0.1% to about 1%, and most preferably about 0.1% to about 0.5% of the specimen volume.

145. The apparatus of claim 119, wherein an additional stabilizing agent is polyethylene glycol at a concentration of about 0.1% to about 5%, preferably about 0.1% to about 1%, and most preferably about 0.1% to about 0.5% of the specimen volume.
